(19) United States
(12) Patent Application Publication
(10) Pub. No.: US 2010/0249765 A1
(43) Pub. Date: Sep. 30, 2010

(54) TRACHEOBRONCHIAL PULMONARY CRYOGENIC THERAPEUTIC METHOD AND APPARATUS

(76) Inventor: Mark H. JOHNSTON, Lancaster, PA (US)

Correspondence Address: BROWDY AND NEIMARK, P.L.L.C.;624 NINTH STREET, NW, SUITE 300, WASHINGTON, DC, 20001-5303, US

(21) Appl. No.: 12/415,469

(22) Filed: Mar. 31, 2009

(51) Int. Cl. A61B 18/02

(52) U.S. Cl. 606/21

(57) ABSTRACT

Disclosed are methods for the cryoablation treatment of a patients tracheobronchial pulmonary system, such as in the treatment of tracheobronchial lesions and lung cancer. The pulmonary system treatment method employs a specially dimensioned dorsal end catheter system that includes a proximal end suitable to provide for the delivery of a cryogenic material to a desired pulmonary area of interest in a patient. The apparatus includes as a part thereof an endoscope dimensioned so as to be suitable for insertion through a patient's trachea. Provided are tracheobronchial cryoablation methods for treatment of benign airway disease, early lung cancer, sarcoidosis, Wegner's granulomatosis, rhinoscleroma, recurrent respiratory papillomatosis (RRP), benign tracheal stenosis, pulmonary dysplasia, neoplasia, vascular lesions, inflammatory lesions and pulmonary and/or tracheal degenerative lesions.

### FIELD OF INVENTION

The invention finds applicability in the filed of cryosurgery where a catheter is used to convey a cryogas to ablate tissue. In particular, the invention finds applicability in the field of cryosurgery in pulmonary tissue and in particular, tracheobronchial tissue.

### BACKGROUND OF THE INVENTION

Cryospray ablation (CSA) is a tool that has been used in a method for freezing diseased tissue. Upon freezing, the tissue evokes tissue necrosis, allowing the regeneration of healthy tissue.

Cryoablation has been described for use in the treatment of gastrointestinal disease. For example, a method for ablation of tissue in the esophagus using a cryogenic gas is described with the use of a specially designed cryosurgical apparatus for gastrointestinal applications in U.S. Pat. No. 7,025,762. The device includes, in some aspects, an inflating means which aids in preventing gas escape of cryogen into the stomach. The device is described as part of a method used in the treatment of a disease of the gastrointestinal tract, such as internal lesions of the esophagus or gastrointestinal tract. A specific application of the gastrointestinal applications of the method is described for the treatment of Barrett's esophagus. It is reported therein that the cold cryogenic gas tends to make the catheter stiff and unmanageable, and at times rupturing the catheter.

In U.S. Pat. No. 7,255,693, a system that provides for electrically heating a catheter for ablation of tissue in the esophagus is described. Heating the catheter is described to maintain the flexibility of the catheter during cryogenic surgery, and to prevent ice formation, which leads to sticking.

In US2007/0276360, a cryosurgical catheter which is heated in order to prevent freezing of an endoscope used in cryoablation on an internal tissue, e.g., the esophagus, is also described.

Lee (U.S. Pat. No. 3,298,371) teaches a cryogenic probe useful in neurosurgery. This patent also describes a electric means for heating the exterior of the probe.

Thomas (U.S. Pat. No. 3,507,283) describes a cryosurgical probe whose temperature is precisely controlled to a desired heat or cold level by employing a heating wire along the external surface of the instrument.

A need continues to exist in the medical arts for apparatus and methods for employing cryogenic surgery in pulmonary applications.

### BRIEF SUMMARY OF THE INVENTION

The present invention, in a general and overall sense, relates to specially designed cryosurgical apparatus for tracheo pulmonary use and methods of using the apparatus for treating tracheo pulmonary pathologies. In particular, the methods employ cryospray ablation in the treatment and rehabilitation of affected tracheo pulmonary tissues.

Among other features and advantages, the tracheobronchial therapeutic methods disclosed herein provide for the safe and effective treatment of pulmonary vascular, neoplastic degenerative and/or inflammatory lesions. By way of example, tracheobronchial therapeutic applications of the present invention may include the treatment of pulmonary tissue lesions through cryoablation techniques, wherein a specially designed cryoablation apparatus is created, such as by modifying the apparatus described in U.S. Pat. Nos. 7,255,693 or 6,027,499, (specifically incorporated herein in their entirety), so as to be suitable for easy insertion to the trachea. In order to accommodate use of the technique for tracheobronchial therapeutic applications without trauma to the surrounding tissue of the trachea, it is advantageous to provide a device that is dimensionally appropriate for insertion into the trachea. The methods according to the present invention may be carried out employing the cryotherapy device described in U.S. Pat. No. 7,255,693, US Patent Publication 2007/0276370, U.S. Pat. No. 7,025,762 or U.S. Pat. No. 6,383,181 (all incorporated herein by reference.).

In the present invention, and recognizing the specific dimensional preferences of a device most suitable for using cryoablation in tracheobronchial therapy, a cryosurgical apparatus may also be employed that is modified so as to preferentially accommodate insertion into the trachea. In these embodiments, a cryosurgical apparatus is provided that comprises a distal end dimensioned for insertion into a patient's trachea. In some embodiments, this cryosurgical apparatus comprises an elongated flexible tubular body having a proximal end, a distal end, and a tubular passageway extending there through for carrying a cryogen from the proximal end to the distal end. In some embodiments, the proximal end may be further described as being adapted to receive a cryogen, while the distal end is adapted to dispense the cryogen to the desired afflicted and/or diseased area of a targeted bronchial, tracheal or other tracheobronchial tissue of the patient. As described in the above-cited patents and published patent application, the cryosurgical apparatus will further comprise a cryogen source.

According to some embodiments of the invention, the apparatus will comprise an endoscope that is specifically dimensioned for insertion into a patient's trachea. This endoscope would preferentially have an inner dimension of about 2.0 mm to about 3.0 mm., or in some embodiments, about 2.0 mm to about 2.5 mm.) By way of example and not exclusion, it is anticipated that the dimensions of a bronchoscope optimally dimensioned for use in tracheobronchial systems would have a scope length of about 870 mm, a channel through which an endoscope (cryoscope) would be included of an inner diameter of about 2.0 to about 3.0 mm in diameter, and a distal outer diameter of about 5.5 mm. to about 6.0 mm. (in some embodiments, a distal outer diameter of about 5.5 mm). (See FIG. 40) The catheter for use in tracheobronchial applications would preferably have a catheter diameter of about 6 to about 7 French (about 2 to about 3 mm in diameter), and a catheter length of about 900 mm to about 1200 mm. (See FIG. 40).

In another aspect, the present invention provides methods for treating tracheobronchial tissues in a number of tracheobronchial system pathologies as well as airway injury generally. By way of example, the methods may be used in the treatment of benign airway disease, treatment of the pleural space of the lung, malignant airway disease (including early and late stage lung cancer), sarcoidosis, Wegner's granulomatosis, rhinoscleroma, recurrent respiratory papillomatosis (RRP), benign tracheal stenosis, pulmonary dysplasia, neoplasia, vascular lesions, inflammatory lesions, emphysema, chronic bronchitis, COPD, asthma, mesothelioma, and pulmonary and/or tracheal degenerative lesions It is envisioned that the tracheobronchial pulmonary cryogenic therapeutic methods of the present invention may be used to treat any number of different pathologies associated with the tracheobronchial system. By way of example, such includes tissue-focused treatment (cryoablation) of any region or regions of the larynx (such as for the treatment of laryngeal lesions (squamous cell carcinomas), polyps of the vocal cords), the pharynx, the trachea, the left and/or right bronchus, or any combination of these. The methods of treating a tracheobronchial system of a patient as described herein may be further described as comprising the steps of providing a cryosurgical apparatus having a distal end dimensioned for insertion into a patient's trachea, inserting the distal end of the apparatus into the trachea of the patient, positioning the distal end within a targeted area of the tracheobronchial system, and dispensing the cryogen to the target area in an amount and for a period of time sufficient to result in tissue ablation, thereby ablating the target area of the tracheobronchial system of the patient being treated.

The cryogen in some embodiments of the method is liquid nitrogen. In some embodiments, the liquid nitrogen is to be sprayed through the catheter via a flexible fiber optic broncoscope (FFB). The cryogen is preferably delivered to the target area of the tracheobronchial system (e.g., the lung) in a substantially radial direction and substantially perpendicular to the axis of the catheter.

In some embodiments, the cryosurgical apparatus comprises a broncoscope dimensioned for insertion into a patients trachea with a scope length of about 870 mm, a channel inner diameter of about 2.0 mm to about 3.0 mm, and a distal outer diameter of about 5.5 mm. to about 6.0 mm. The endoscope is inserted through the channel identified in FIG. 40 (“Instrument Channel Outlet”).

Certain tissues may have variable sensitivity to cryotherapy and this difference may be exploited in treatment (Sheski, Clinics in Chest Med. 1999).

A completely automated system with sensors and a microprocessor are employed for performing cryosurgery. It is an important preferred feature of the present invention that the spray be conducted in such a manner as to allow constant visualization by the physician of the tissue treatment as it occurs. If the temperature of the lens at the distal end of the endoscope drops precipitously at the start of the liquid nitrogen spray, the moist air of the esophageal environment or tracheal environment or the air of the catheter which has been blown out ahead of the nitrogen flow will condense on the lens, thereby obscuring the physician's view of the operative site. This can be substantially avoided by means of a suction pump which will immediately suck out the moist air which is present prior to the arrival of the liquid nitrogen spray or cold nitrogen gas. Because of this pumping out of the moist air as the spray commences and the replacement with extremely dry nitrogen gas, substantial amounts of moisture will not form on the lens during the procedure, allowing an excellent view of the operative site by the physician during the procedure.

This condensation effect is augmented by the fact that the catheter itself is preferably not wrapped in additional insulation. This causes the temperature of the nitrogen gas exiting the catheter at the distal end to be relatively high at the beginning of the spraying operation and gradually cooling as the catheter cools. In the tests conducted in the esophagus of pigs, often 10-20 seconds were necessary before significant freezing was seen through the endoscope. If the catheter is substantially insulated, the interior of the catheter will cool much more quickly as it will not be picking up heat from the outside. With this insulated catheter, it is to be expected that the liquid nitrogen would be sprayed onto the tissue almost immediately, causing much faster freezing and, thus, allowing less control on the part of the physician.

Another reason that the lens does not fog or frost in the present invention is that the esophagus is flushed out with nitrogen gas, which is extremely dry. The nitrogen gas is moisture free because the liquid nitrogen is condensed out of atmospheric gases at a temperature −196° C. colder than the temperature at which moisture is condensed out.

The combination of relatively warm, and completely dry nitrogen gas, together with suction flushes all moist air from the esophagus or trachea. As the temperature of the gas entering the esophagus or trachea falls, so does the surface temperature of the camera lens. Ordinarily at that time the lens would be cold enough to condense moisture and fog, however, since the esophagus is dried out (in contrast to its usual highly moist state) there is no moisture to condense. Thus, the lens stays un-fogged and un-frosted and continues to provide a clear view of the operation. On the other hand, if the esophagus is not vented with suction and/or the esophagus or trachea is not preliminarily flushed with dry nitrogen gas (perhaps because the catheter is insulated, lowering its heat capacity, and/or the nitrogen delivery pressure is too high), then the lens is likely to fog or frost and the physician cannot operate effectively.

In order to deal with the moist air problem, there is supplied in the preferred embodiment of this invention a nasogastric tube. During the cryosurgical procedure the nasogastric tube is inserted prior to inserting the endoscope. The nasogastric tube, when connected to a pump, can serve to evacuate moist air from the esophagus and/or trachea prior to cryosurgery. With moist air removed, the TV camera lens is not obscured by fog and the physician can perform cryosurgery with an unobstructed view. Alternatively, if fogging occurs during cryosurgery, the nasogastric tube and pump can be used to evacuate the esophagus and/or trachea.

In some embodiments, the composition of the catheter or the degree of insulating capacity thereof will be selected so as to allow the freezing of the mucosal tissue to be slow enough to allow the physician to observe the degree of freezing and to stop the spray as soon as the surface achieves the desired whiteness of color (cryoburn). The clear observation results from the removal of the moist air and sprayed nitrogen by the vacuum pump; in combination with the period of flushing with relatively warm nitrogen prior to application of the spray of liquid nitrogen which is caused by the relative lack of insulation of the catheter. Preferably, the catheter has a degree of insulation which permits at least five seconds to pass from the time said means for controlling is opened to the time that liquefied gas is sprayed onto the mucosa. As a preferred embodiment, an electrically heated catheter is described herein.

An electronic monitoring and recording system is to be used during cryosurgery. The electronic components of the system comprise a temperature sensor or probe and timer. Also connected to the monitoring and recording system are the foot-pedal for actuating the solenoid and recording console. An electric power cord runs from solenoid to control box.

The temperature sensor is thin and can be inserted into the esophagus or trachea beside the catheter. In some embodiments, the temperature sensor and catheter can be inserted separately or as an integral unit of sensor and catheter combined, or alternatively the sensor can be inserted through an extra lumen of the endoscope to come in contact with the tissue of the esophagus or trachea. The temperature sensor sends temperature readings to the electronic monitoring and recording system for processing and recordation.

The liquid gas flow is started by actuating solenoid foot-pedal and ends with release of the solenoid foot pedal The electronic monitoring and recording system records the times at which cryoburn starts and ends. Temperature in the context of time will be recorded for the cryosurgery. This recordation allows for better data acquisition and documentation.

There is an automatic cut-off if a time or temperature limitation is exceeded. In the event of a cut-off, the electronic monitoring and recording system can be reactivated by pushing the reset button. Current time and temperature readings are presented in the windows as LED numbers. The windows in the system will indicate total time; shut-down time; cryotime; cryotime set; and temperature. Within the main console of the electronic monitoring and recording system is a printing unit which prints and records the time and temperature during the cryoburn. Every event is recorded, e.g. time, on and off, temperature, etc.

The electronic console can be preprogrammed to be patient specific.

Kit Supplying Components of the Invention. The components or paraphernalia required to practice the method of the present invention may be packaged and sold or otherwise provided to health-care providers in the form of a kit. The kit is preferably sealed in a sterile manner for opening at the site of the procedure. The kit will include the catheter, having the spray means at one end, as well as a means for connecting the catheter to the source of liquefied gas. This means for connecting may be a simple luer connection on the opposite end of the catheter from the spray means. However, the term “means for connecting said catheter to a source of liquefied gas” is intended to include any other device or apparatus which allows the catheter to be connected to the gas source.

Many of the components of the cryosurgical system are conventional medical appliances. For example, the endoscope is a conventional medical appliance and would not necessarily have to be supplied as part of a kit. One of the components to be supplied in a kit or sterilized package is a combined catheter-bleeder vent. Also, the heated catheter assembly would be supplied in a kit or sterilized package.

It is envisioned that supplying the heated catheter and vent unit will be as a separate item. In this way, the unit can be supplied in a sterile packet or kit to be used with existing equipment found in hospital operating rooms. The kit may contain a nasogastric tube, or the kit could contain only a heated catheter unit.

The means for controlling the flow of liquefied gas to the catheter is also preferably present in the kit and may be connected to or may be part of the means for connecting the catheter to the source of liquefied gas. For example, the connector may contain a valve therein or the valve may be a separate element connected between the connector and the catheter or between the connector and the nitrogen source. The connector besides being connected to the source of gas can also be a connector to the source of electricity.

The kit will also optionally contain the means for withdrawing gas, such as a tube and a means connectable to the tube for withdrawing gas from the tube. Such means connectable to the tube for withdrawing gas may be a vacuum pump or any other device or apparatus which will accomplish the function of withdrawing gas from the tube. The vacuum pump is optionally omitted from the kit as a source of vacuum is often found in hospital rooms in which such a procedure is to take place.

The means for blocking the lumen is also optionally present within the kit. Thus, for example, the kit may contain a balloon catheter or any other device or apparatus which can accomplish the function of blocking the lumen when in use.

The term “container” or “package” when used with respect to the kit is intended to include a container in which the components of the kit are intended to be transported together in commerce. It is not intended to comprehend an entire procedure room in which the individual components may happen to be present, an entire vehicle, a laboratory cabinet, etc.

Pressure During Cryosurgery. In an embodiment of the invention, the bleeder valve has been found to be unnecessary so long as low pressure can be maintained by other means. In some embodiments, a cryoburn is carried out without the need for a bleeder valve. In this embodiment with the tank pressure at 45 psi and the catheter being a 9 french, the cryo-procedure took 4 minutes and 50 seconds. With a 10 french catheter using 45 psi, the cryo-procedure took 2 minutes and 50 seconds to achieve a cryoburn temperature. With the bleeder valve, it takes 10-20 seconds to achieve cryoburn. The ideal low pressures operative for this invention should be in the range of 3-45 psi. The most ideal pressure is determinable by those skilled in the art.

Regarding pressure, 40 psi is preferred, the cryogenic spray will function at higher pressures. The system could be made to work at tank pressures as high as 300-400 psi by adjusting the size of the bleeder line and by using a larger size catheter. Note, however, that tip pressure is only one factor to be considered for producing cryoburn. Other factors to consider are size of catheter and length of time of application. Certain clinical conditions may require differing pressures and differing time of cryoburn. The nozzle or tip pressure for cryosurgery should not be so high as to puncture any internal organ and optimum nozzle pressure can he determined by those skilled in the art. A low pressure that would be better tolerated for in vivo tracheobronchial pulmonary use would theoretically be similar as in the GI tract. However, it is anticipated that a lower pressure would be even better accommodated for application to the trachea and pulmonary-bronchial tissues. The cryosystem could function at significantly higher nozzle pressures by adjusting other factors of the protocol. Significantly higher nozzle pressures would be operative if the treatment exposed the tissue to shorter cryoburn exposure time. The higher pressures may necessitate the need for a vacuum line to remove the excess volume of nitrogen introduced into the body cavity.

In the future, technology may reduce the size of the components of the endoscopic. This would allow additional diameter for the catheter. If the diameter of the catheter is increased, the flow of the cryogen could also be increased without affecting the treatment parameters. Potentially, the catheter could be used along side of the endoscope rather than through the lumens of the endoscope. Then the size limitation of the catheter could be modified.

Additionally, the holding tank could be stored at much greater pressures. The higher the storage tank pressure, the less nitrogen bleed off that will occur, resulting in a lower loss of nitrogen during storage. The temperature of the liquid nitrogen stored at pressures higher than 22 psi is warmer than that of the liquid nitrogen stored at 22 psi. At 200 psi (this is the highest pressure tested) the liquid nitrogen is still cold enough to deliver a cryoburn.

The high-pressure tank can be staged in any conceivable manner. A 700 psi storage tank could be staged down by altering the size of the bleeder, by altering the size of the catheter, or by adding additional bleeder lines. A 700 psi flow to 3-5 psi can be accomplished in a number of ways as understood by those skilled in the art.

Nozzle pressures of catheters have been examined and found for tank pressure of 22 psi and a 9-French catheter the nozzle pressure is 2-3 V₂ psi; and for tank pressure of 22 psi and a 10-French catheter the nozzle pressure is 3.2-5.9 psi.

A bleeder valve is not absolutely essential to this invention since low pressure cryoablation can be carried out through low head pressure in the storage tank or through selection of the proper inner diameter of the catheter. Based on studies carried out with the bleeder valve embodiment a shorter time period is required for cryoburn.

A convenient and preferred means of supplying the cryogenic gas under pressure and in liquid form would be to employ a compressor to compress the gas to be used with the catheter before it is to be used in cryosurgery.

Cryoburn Conditions. Preliminary test results show that a 30 second “cryoburn” time was adequate to ensure the appropriate tissue destruction, and thus appropriate cellular healing of damaged tissue (this conclusion was based on a 30 day follow up period).

“Cryoburn” is a term defined by the instance that the normally “pinkish” esophageal or tracheal tissue turns white (much like freezer burn). A range for the “cryoburn” time could be 5-10 seconds to 2 minutes or more depending on the substrate to be treated.

Due to the nature of the system, “cryoburn” does not immediately occur, but rather requires that the entire fitting and catheter system become cool. Typically this required approximately 20-30 seconds from the time that the solenoid foot pedal is depressed, and liquid nitrogen is allowed to flow from the tank.

During animal testing the approximate temperature that cryoburn was first observed was at approximately −10° C. The temperature range for cryoburn would be approximately −10 to −90° C.

In carrying out the procedure, a nasogastric tube is first inserted into the esophagus, after which an endoscope is inserted. Optionally, attached to the endoscope will be a temperature probe to sense the temperature and report the temperature to the recording console. Once the nasogastric tube, endoscope and temperature probe are in place, the catheter attached to the gas supply will be inserted into a lumen of the endoscope. Before liquid gas is supplied, the esophagus and/or trachea is ventilated using the nasogastric tube to remove moist air (if required). With the moisture evacuated and the endoscope properly positioned, gas can be supplied to the catheter by actuating the solenoid with foot pedal. Once the solenoid is actuated gaseous nitrogen and then a spray of liquid nitrogen will come from the tip of the catheter. The cryoburn will generally last for 30 seconds to two (2) minutes. For tracheobronchial pulmonary use of the apparatus and/or method, a nasogastric tube need not be inserted into the trachea as part of carrying out the methods. Alternatively, a significantly smaller diameter tracheal suction tube may be utilized instead of a nasogastric tube.

In further developing the cryogenic spray system, several positive advantages are envisioned in over-exposing the esophagus or trachea to the cryoburn. For example, the scarring that occurs could be helpful for patients that have chronic reflux. There are currently a number of techniques that work to “tighten” the lower esophageal sphincter. The scarring that occurs during over exposure in the cryosurgical method of the disclosed invention could be an additional treatment of chronic reflux.

Studies. The cryospray was used in studies to assess the efficacy and safety of this device in mucosal ablation in the distal esophagus of swine. The catheter was a long 7 Fr ERCP-like catheter placed through the biopsy channel of an Olympus GIF-100 endoscope. The swine were sedated using telazol and xylazine given intravenously. General anesthesia was not necessary. Liquid nitrogen was sprayed on the distal 2 cm of the esophagus in 16 swine under direct endoscopic observation until a white “cryo-burn” appeared, usually within 10-20 seconds. Duration and location of the spray were varied to assess histologic response and depth of “cryo-burn”. The swine were then re-endoscoped on days 2, 7, 14, 21 and 30 to obtain biopsies from the injury site, assess mucosal ablation and re-epithelialization. All swine were then euthanized and underwent necropsy.

Freezing of the esophageal mucosa and/or mucosa of the tracheobronchial pulmonary system tissue may be recognizable by a white “cryo-burn” with sharply demarcated margins. This was followed by slow thawing within minutes and then mucosal erythema. Sixteen swine underwent hemicircumferential to circumferential cryotherapy of their distal esophagus varying the duration of “cryo-burn” from 10-60 seconds. Blistering and sloughing of the superficial mucosa occurred within 2 to 7 days of the cryospray. Mucosal damage occurred only at the cryo site. Biopsies 48 hours after cryospray consistently demonstrated coagulative necrosis involving the mucosal layer and biopsies 30 days after cryospray consistently demonstrated complete re-epithelialization of the injured area.

These studies on living swine, which are a valid model of the human system, establish that cryotherapy spray of liquid nitrogen via upper endoscopy is a simple technique capable of inducing controlled superficial mucosal damage with complete healing in the esophagus and other areas, such as for the treatment of the tracheobronchial pulmonary system (pulmonary system).

The low-pressure device (FIGS. 28 and 29) described by Johnston (Gastrointest. Endoscop. 1999) and colleagues, uses liquid nitrogen in a specially designed system that operates at a maximum of 30 psi. The catheter, 10F, is multilayered. Its outer sheath is coated with a special polymer that can be warmed during the cryo application, thus maintaining catheter pliability and the unique ability to operate at a very low pressure. This device also uses a foot pedal for control of gas release and a temperature probe for monitoring mucosal temperature during the cryo application. With this delivery system, the depth of injury is controlled by manipulating 3 parameters: the duration of cryo application, extent of cryoburn viewed endoscopically, and the temperature of mucosa at the time of application. These parameters are monitored via a special software program and device that is part of the cryogenic system (FIG. 29).

Low Pressure Cryo-therapy Device. Four separate phases of animal research have been conducted with the low-pressure device. In the first phase, twenty swine underwent cryoablation of the distal 2-3 cm of their esophagus with liquid nitrogen in either a hemicircumferential or circumferential pattern and were followed for one month post-cryotherapy (Johnston, Gastrointest. Endosc. 1999). In the second phase, 8 swine were treated hemi-circumferentially to the distal 3 cm of the esophagus and followed for 90 days. In the third phase of experiments, 4 swine underwent endoscopic ultrasound (EUS) of their esophagus pre-cryo, immediately post cryo and then at 48 hours, 7 days and 14 days to assess the effects on the esophageal wall. In the final phase, one swine was treated in different locations with Argon plasma coagulation (APC), Multi-polar electrocoagulation (MPEC) and cryotherapy. The lesions were then compared both endoscopically and microscopically.

Additional features and advantages are described herein, and will be apparent from the following Detailed Description and the figures.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 is a perspective view of the heated catheter assembly. Part of the catheter is broken away for ease of illustration.

FIG. 2 is a cross-section of the heated catheter assembly, taken along 2-2 of FIG. 1, with the hub portion broken away.

FIGS. 3, 5 and 6 are views taken along cross-section 5-5 of FIG. 2 to show components forming the heated catheter.

FIG. 4 is a cross-section taken along 4-4 of FIG. 2.

FIGS. 7-11 illustrate the steps taken to construct the heated portion of the catheter. These views are cross-sections taken longitudinally as 2-2 in FIG. 1.

FIGS. 12-18 show the method for assembling the hub portion of the heated catheter.

FIG. 19 is a perspective view of the gas and electric connector subassembly.

FIG. 20 is a cross-section thereof taken along lines 20-20 of FIG. 19.

FIGS. 21-27 illustrate means by which the catheter is jointed to the gas and electric connector subassembly, with FIGS. 21-23 being longitudinal cross-sections of the gas and electric subassembly and top of the hub.

FIG. 24 is a plan view of the gas and electric connection joined to the hub. The arrows show the direction for joining the components.

FIGS. 25 and 26 are cross-sectional views taken off of 25-25 and 26-26 of FIG. 20.

FIG. 27 is an enlarged longitudinal cross-section of the heated catheter.

FIG. 28 is a photograph of the heated catheter.

FIG. 29 is a photograph of the complete low pressure spray cryotherapy device.

FIG. 30 is a photograph of an endoscope that can be used in cryotherapy.

FIG. 31 through 38 are photographs of cryoburns and histology resulting therefrom.

FIG. 39—Method of pulmonary cryogenic ablation with a cryoablation device according to the invention suitable for entrance into the trachea. The trachea-modified catheter is passed through a bronchoscope and into the lung of the patient/animal.

FIG. 40—Tracheobronchial Pulmonary Cryogenic Apparatus. (Left Panel) Scope length: 870 mm, Channel inner diameter: 2.0 mm, Distal outer diameter: 5.5 mm; (Right Panel) Scope Tip (cross section) showing the Instrument Channel Outlet (endoscope), and optionally a Light Guide lens, and an Objective lens.

### DETAILED DESCRIPTION OF THE INVENTION

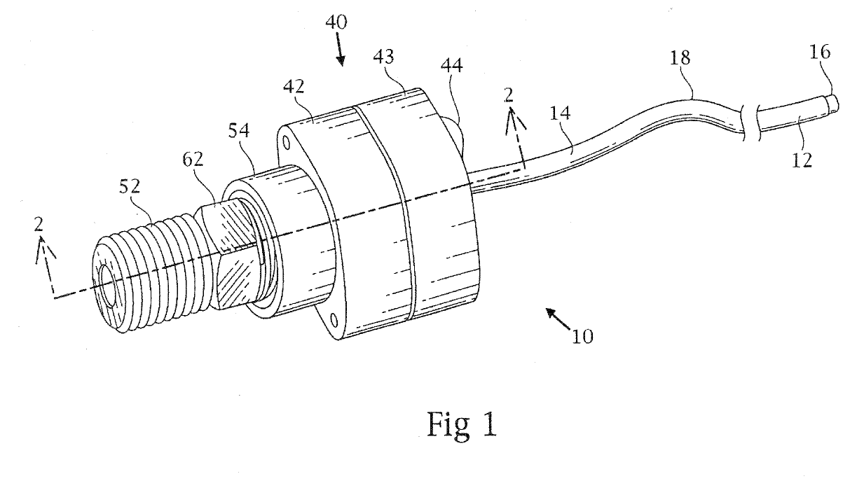

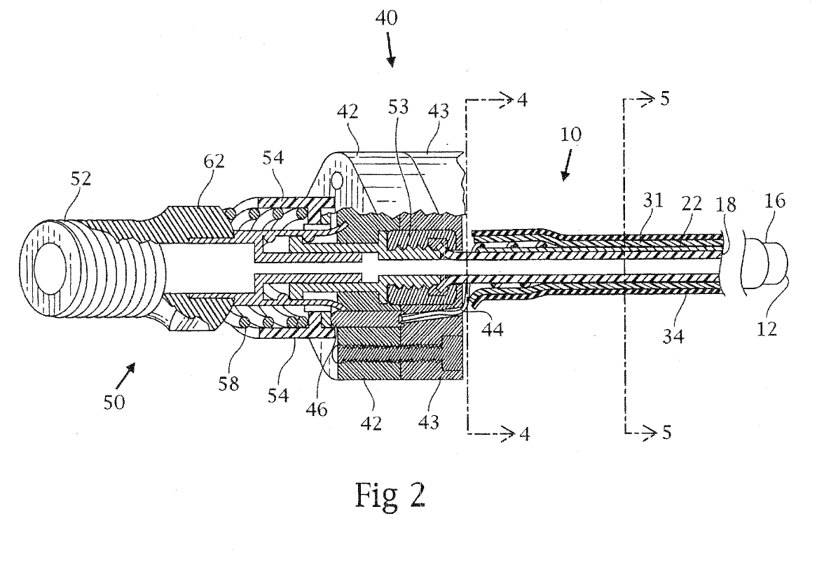

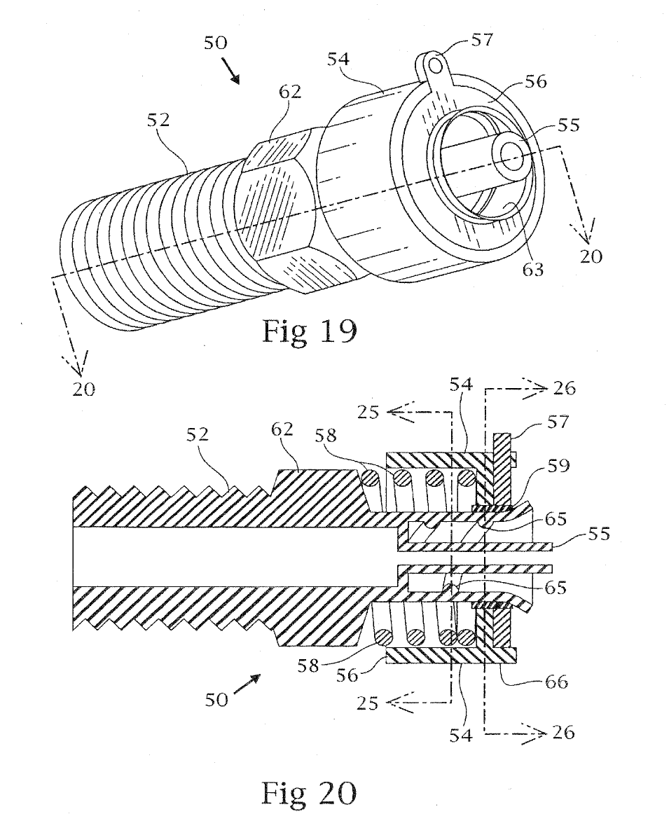

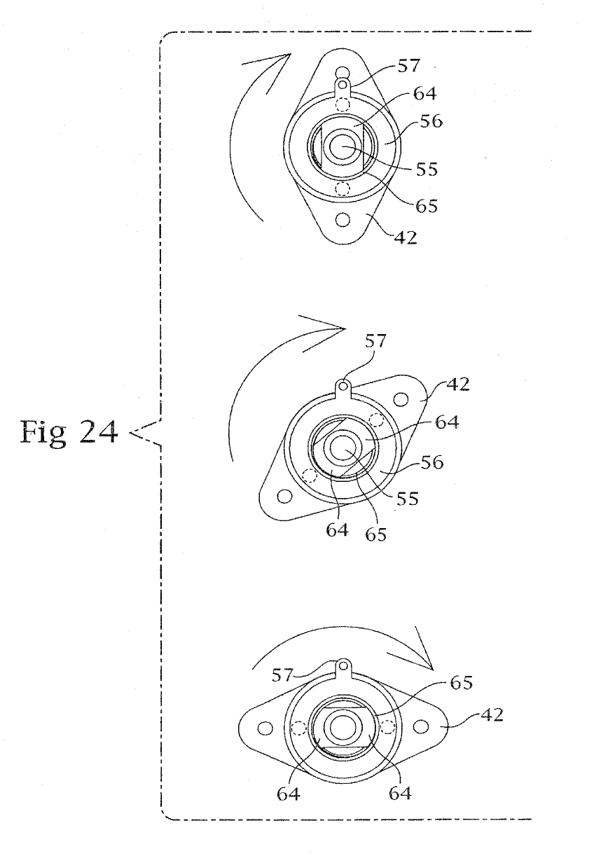

With reference to FIGS. 1 and 2, a heated catheter assembly 10 has a catheter 18 with a distal end 12 and a proximal end 14. As part of the catheter assembly 10 there are a hub 40 having a top portion 42 and a base 43. The top portion 42 of the hub 40 has a gas and electric connector subassembly 50 for attaching the gas line and two contact points for making electric contact with the luer lock and threaded gas nipple 52 (described more fully in FIGS. 19-27).

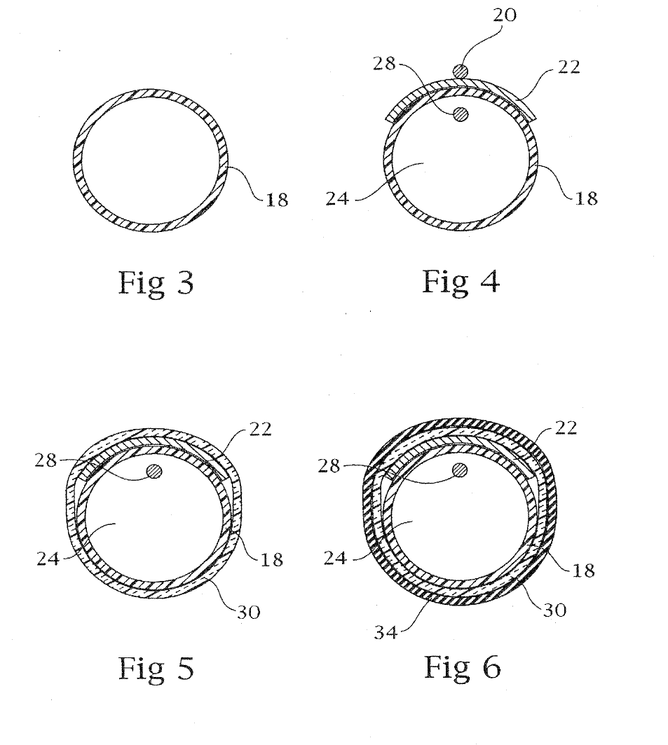

Referring to FIGS. 3-6, the order for constructing the heated catheter is shown in cross-section. Catheter 18 is shown in FIG. 3. FIG. 4 describes the catheter 18, with internal copper wire 28, the external copper wire 20 outside of the catheter and copper foil 22. Copper wire 20 being attached to copper foil 22. Wire 28 runs the length of the internal portion of the catheter 18 and exits at the distal end where it is held in place by a hypodermic tube or stainless sleeve 38 (see FIG. 9). The stainless sleeve 38 presses over the wire 28 exiting the distal end of the catheter 18 to sandwich the wire between the catheter 18 and stainless sleeve 38. With reference to FIG. 5, an electrodag coating 30 covers part of the catheter, that is, the electrodag covers a portion of the catheter contacting a portion of the conductive foil (as explained more fully below). The electrodag coating is a conductive coating and is an integral part of the heated catheter. Finally in FIG. 6 a parylene (dielective insulator coating) coating 34 covers the electrodag coating 30.

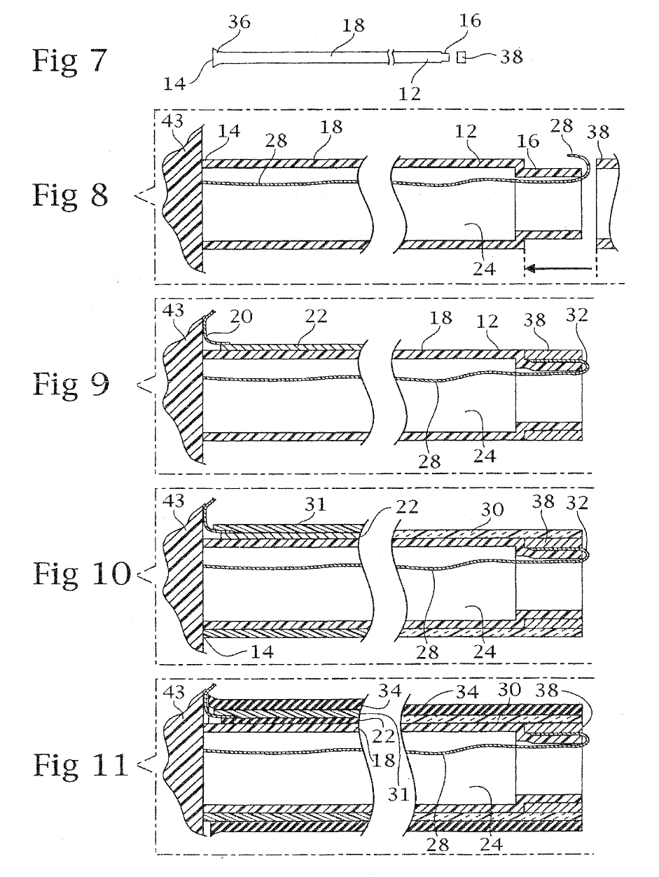

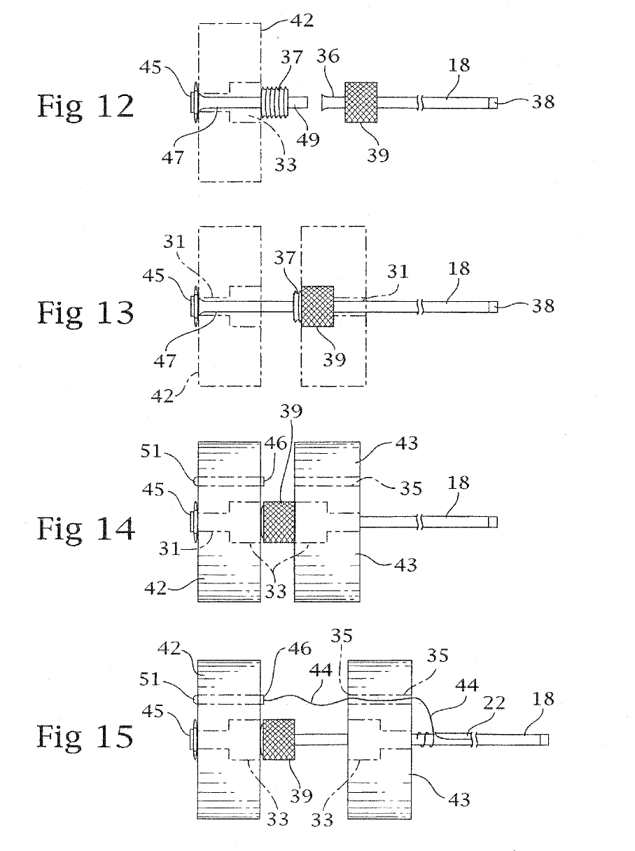

With reference to FIG. 7 catheter 18 has a flared end 36 and stainless sleeve 38. The flared end 36 of the catheter allows for a better seal between the proximal end of the catheter and gas supply channel as more fully explained in FIGS. 12-15 and 27.

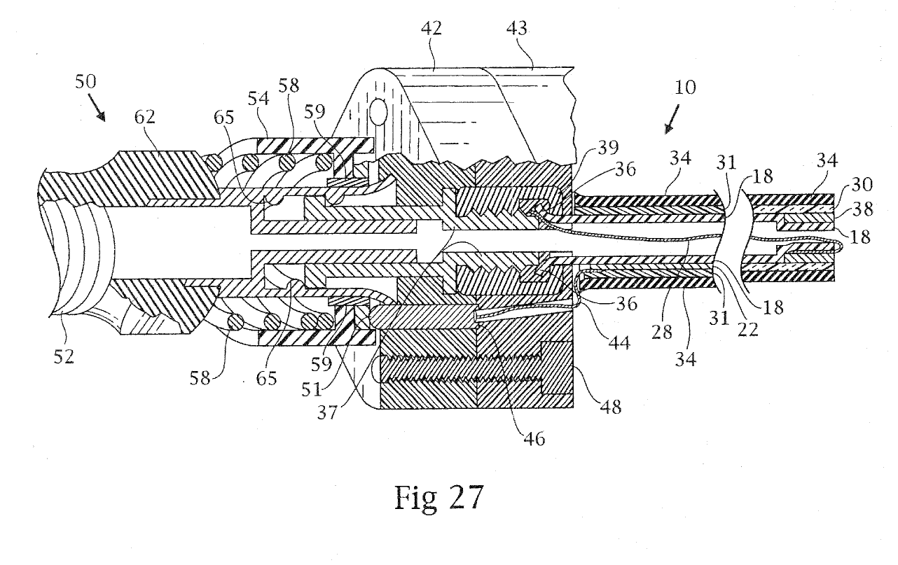

With particular reference to FIGS. 8-11, the heated catheter in longitudinal cross section is shown with part of the hub base 43 broken away. Exemplary of the embodiment of this invention is an 84 inch PTFE basic catheter 18 which has an ⅛ inch groove 16 at the distal end 12 (FIGS. 7 and 8). The catheter 18 is etched (not shown) for bonding. With reference to FIGS. 9-11, a copper wire 28 runs the length of the catheter on the interior 24. The copper wire 28 runs out through a channel in the groove 16 and is folded over the distal end 12 of the catheter to the exterior 32 as best shown in FIG. 9. An ⅛ inch stainless sleeve 38 is press fit over the wire 28, covering the groove 16 completely and securely holding wire 28 in place. The proximal end 14 of the catheter 18 is flared 36 to 0.130″ best shown in FIG. 7. Note particularly an insulating coating 31 covers the foil 22. This insulating coating extends only over a portion of the catheter and is covered by the parylene coating which covers the entire catheter. As shown in FIGS. 12-13, the wire exits the proximal end 14 of the catheter 18 and comes in contact with the post of the luer 37 and then the compression nut of the luer 39 is tightened, locking the catheter 18 and the wire 28 in place, as best shown in FIG. 27.

Note that a copper foil strip 22 is placed longitudinally at the proximal end 14 of the catheter 18. Referring to FIGS. 10 and 11 once the copper foil 22 is in place on the catheter 18 a short heat shrink insulating layer 31 goes from the hub base 40 to cover the copper foil (conductive strip) 22 and a short portion of the electrodag coating 30. The electrodag coating extends from a short portion on the copper foil to the distal end of the catheter and finally a parylene coating (dielectric insulator) covers the hub and catheter portions of the heated catheter, except for the female metal gas orfice 45 and hub contact pin 51. The electrodag coating is a conductive coating containing metal graphite and silver or any conductive material in an epoxy resin and is an integral part of the heated catheter. For purposes of this invention conductive foil 22 is copper, but any conductive material would be operative. The heat conducting strip 22 as disposed on the catheter has proximate end and a distal end. The distal end of the heat conducting strip is in contact with the electrodag coating. The proximal end of the conducting strip is attached to wire 44 (best shown in FIG. 27). The heat conducting strip, the thin wire and electrodag coating when attached to the power source produce the heat for the catheter. In a typical example the outer coating is heat shrink and will cover the entire foil section. As an example, the electrodag extends from the distal end of the foil, covering 1.5″ of the foil, to the tip of the catheter, covering the stainless sleeve on the end of the catheter and is in turn covered with dielectric insulator 34.

With particular reference to FIGS. 12-15, the manner for attaching the catheter 18 to the hub 40 is illustrated. FIG. 12 describes hub top 42 (in dashed lines) fixedly attached to the thread male portion 37 of the luer. In FIG. 12 there is illustrated a tube 47 having at its left end a gas intake orifice or luer 45, in its middle there is fixedly attached the male thread 37 and its right end there is a male catheter connector 49 which engages the flare 36 of the catheter in the female portion 39 of the compression nut. On tube 47 there is fixedly attached hub top 42 having gas intake orifice 45 and male luer 37 exposed. Specifically in assembling the catheter to the hub a wire 44 attached to copper foil 22 at the proximal end 14 of the catheter 18 the male end of tube 49 and female of luer 39 are joined and the wire is snugged tight in the luer as best shown in FIG. 27. Referring to FIGS. 14-16 the means for attaching wire 44 from the copper foil 22 to the post 46 attached to the internal portion of hub top 42 are shown. Note that wire 44 is attached at one end to the copper foil and runs from the copper foil through a channel in top 43 to attach to post 46 (e.g., by solder). Post 46 runs through the top of the base 42 to form post 51 which contacts electric ring 56 best described in FIG. 27. With reference to FIGS. 14-16, the top 42 and base 43 of the hub have hollow compartments 33 for retaining the luer.

Referring to FIGS. 15-18: At the proximal end of the catheter 18, an electrical wire 44 is soldered to the proximal end of the copper foil 22, the electrical wire 44 wraps a few times around the catheter 18 and enters the base of the hub 43, and through channel 35 to contact post 46 at the top of the hub 42. Note that contact post 46 extends through top 42 and becomes hub contact pin 51 which contacts ring 56 on hub connector 54 (described in detail in FIGS. 19-24). Electrical contact is made through metal luer and outside post.

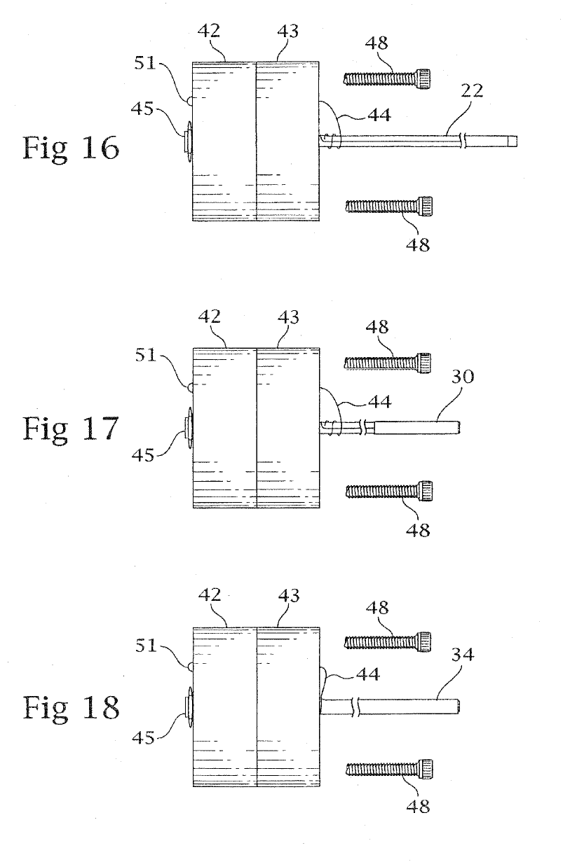

As an example for assembling the heated catheter, 53 inches from the distal end 14 of the catheter 18 a short section of copper foil 22 runs along the exterior of the catheter 18 to the proximal end 14 and a connecting wire 44 runs from the copper foil through a channel 35 in the base 43 of the hub 40. Wire 44 attaches to the copper post 46 of the top section 42 of the hub 40 (FIG. 15). The copper post 46 is connected to a gold plated contact pin 51 on the surface of the hub 40. The base 43 mates to the top section 42 and is secured in place by two screws 48 (FIG. 16). The distal most 55 inches of the catheter is covered with electrodag coating 30, which covers all of the distal stainless steel hypodermic tube and two inches of the proximal foil (best shown in FIG. 10). Heat shrink coating 31 is applied 51 inches from the distal end and extends to the hub, completely covering the copper foil (FIG. 11). The Parylene or heat shrink coating covers the exterior of the catheter over the entire surface including the electrodag coating (FIG. 11). The outer coating is a dielectric or insulating coating. Advantageously, a Parylene film is used as the outer insulating coating because it can be formed in extremely thin layers. The flare 36 in the catheter tubing creates a positive fluid seal with the luer, while the hub 40 serves as the electrical connection for the wire on the interior of the catheter and from the foil 22 running the proximal length on the exterior of the catheter tubing. The interior of the hub houses a conductive material, while the outside of the hub is an insulator. The hub and the distal most portion of the catheter both maintain a zero potential electrically.

With reference to FIGS. 19 and 20, there is shown the gas and electrical connection subassembly 50 having a gas connection threaded male gas nipple 52 for receiving the cryogenic gas. The gas nipple is joined at an end to a spring actuated hub connecting means 54 which contains a spring mechanism 58 for insuring a secure attachment. Referring specifically to FIGS. 19-20, the spring actuated hub connecting means 54 has a male member 55 for connecting to the female gas inlet 60 associated with top of the hub 42. Connector 54 also had therein an electric contact ring 56 for contacting pin 51 on the hub top 42, as well as a tab 57 on ring 56.

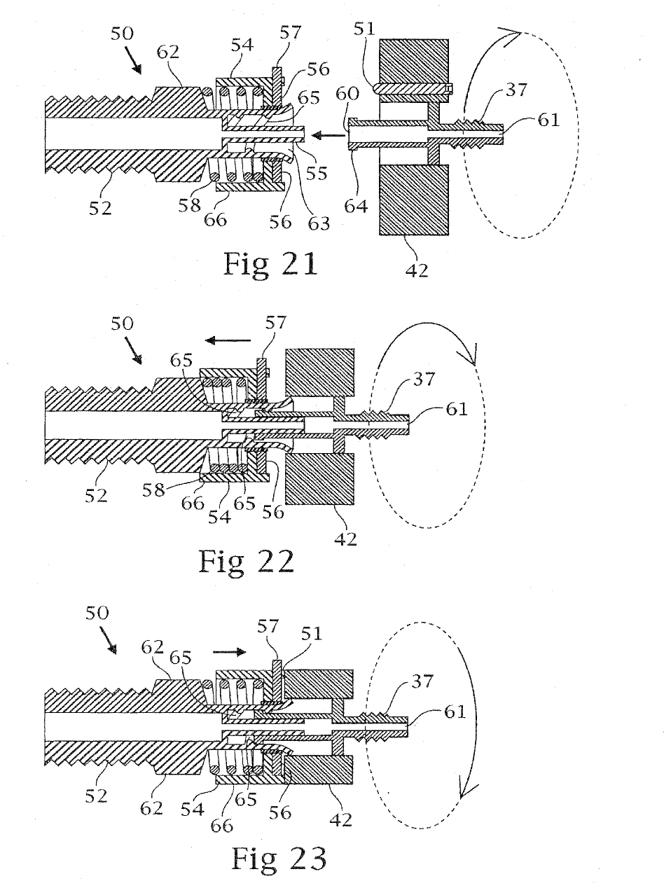

An important element of the electric and gas connection is the spring-loaded hub connector 54 shown in detail in FIGS. 20-24. Note that the hub connector has an internal spring 58 which is compressed by pushing on the sides of connector 54; connector 54 is joined to the female gas inlet 60 of the hub by inserting the male member 55 of connector 54 into female gas inlet 60, compressing the spring 58 in the connector by pushing on the side of the connector. While in the compressed state, male 55 and female 60 are jointed and tabs 64 on the female inlet 60 are inserted into threaded 65 annular opening 63 and turned a half turn so that the tabs 64 fully engage thread 65 as shown in FIG. 21-24. Note particularly that in FIG. 21 springs 58 are not compressed; during engagement the spring 58 is compressed and upon engagement the spring 58 is released assuring a secure attachment. Tabs 64 are inserted into annular threaded opening 63; and the spring is released when tabs 64 and threads 64 are fully engaged. This spring loading insures a secure fit for both gas and electric connections. Note that the spring is retained in the hub connector 54 by abutting an end of the nipple 52 and housing 66.

With reference to FIGS. 21-24, an elegant arrangement for securely joining the gas and electric subassembly 50 to the female gas inlet 60 of the hub top 42 is described. This joining depends upon tabs 64 on either side of female gas inlet 60 entering threaded annular opening 63. Once tab 64 enters said opening 63, the hub connector 54 is retracted, and tabs 64 are allowed to enter the threaded annular opening 63. The tabs 64 which are integral to hub 40 are turned by twisting the hub 40 a half of a turn so that the tabs 64 engage thread 65 in annular opening 63. Once the tabs 64 and thread 65 are engaged, hub connector 54 is released allowing spring 58 within the connector to fixedly attach the hub 40 to the hub connector 54. In FIGS. 21-23 the arrows are intended to illustrate the tabs 64 entering annular opening 63 to engage thread 65.

With reference to FIGS. 21-23, note also that the mating of the gas and electric subassembly 50 with the hub gas inlet 60 is described. On the opposite end of the gas inlet 60, there is a gas outlet 61 having disposed thereon the threaded member 37 of the luer. There is also fluid seal and electrical components for contacting the heating components of the catheter to the electric power supply.

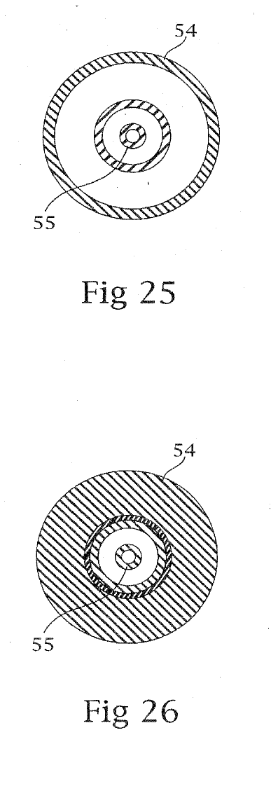

With reference to FIGS. 25 and 26, a cross section taken along lines 25-25 and 26-26 are described to show the male portion 55 of connector 54.

FIG. 27 is an enlarged cross section of the heated catheter, the components of which have been described.

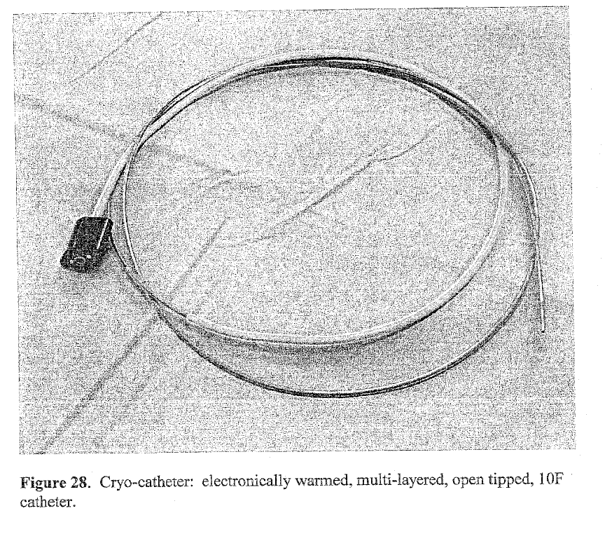

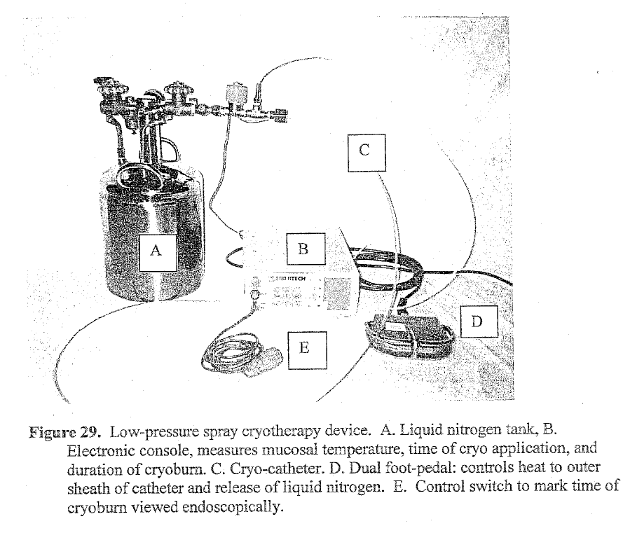

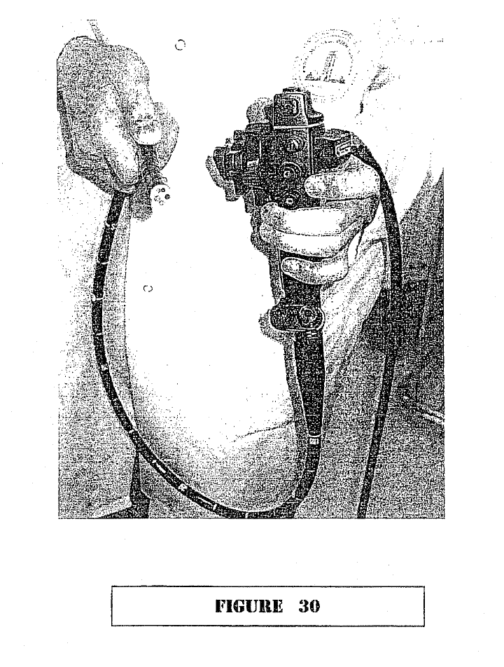

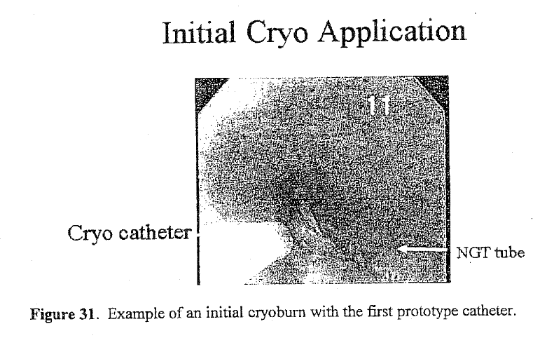

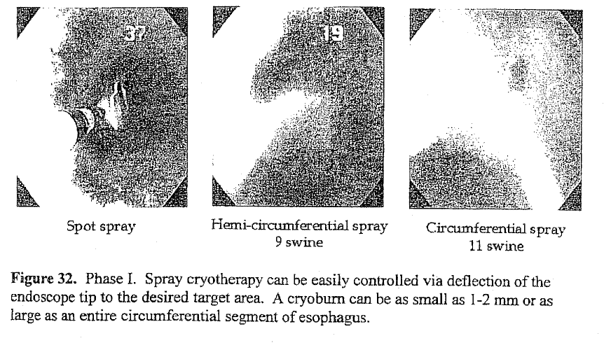

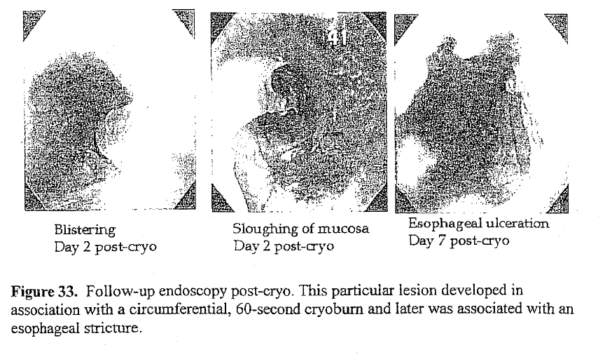

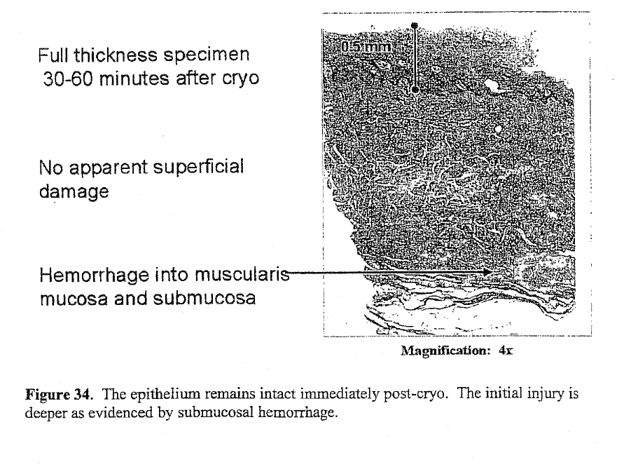

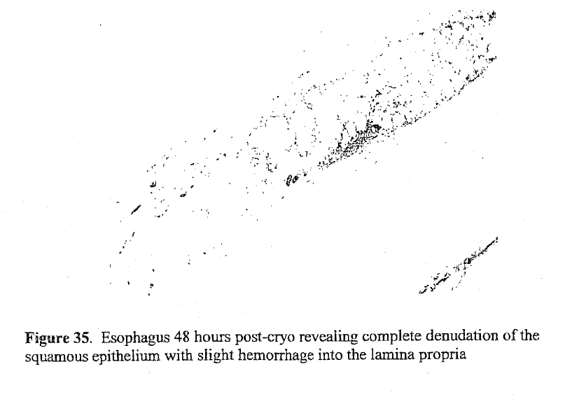

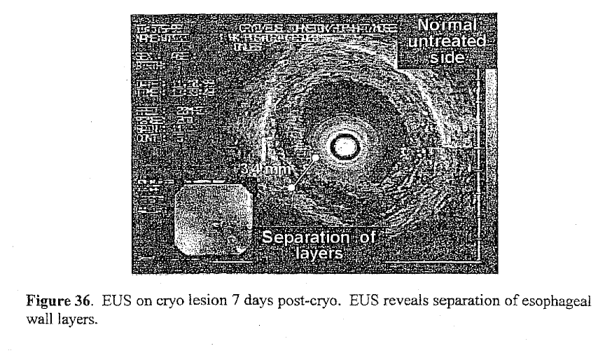

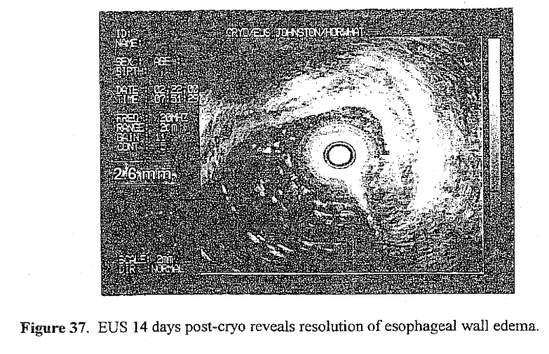

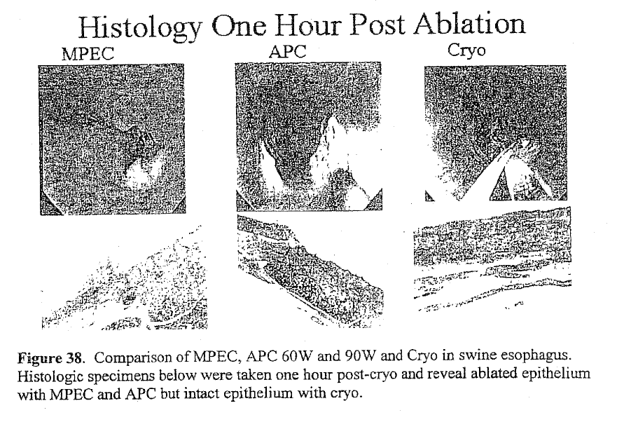

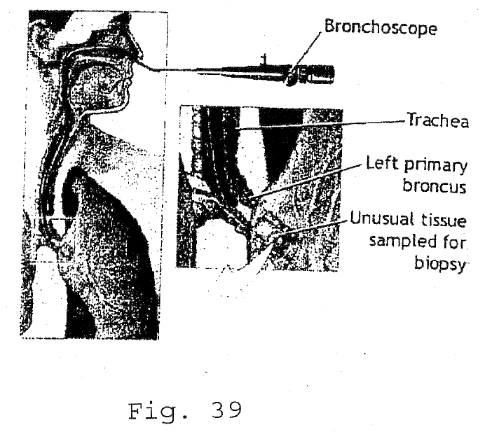

FIGS. 28-38 are pictorial vies bearing self-explanatory legends.

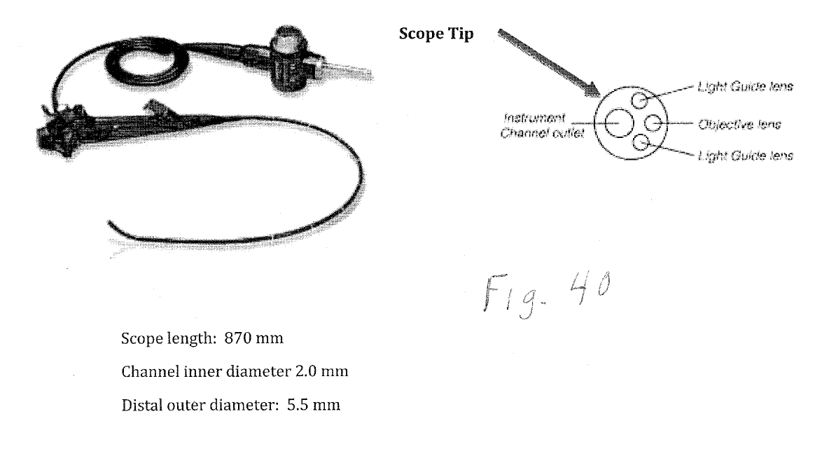

FIG. 39 provides pictorial views showing an endoscope introduced into a patient's trachea according to the invention.

The male fitting connected the solenoid mates with the hub 40 to provide both a fluid seal and an electrical connection. The center of the fitting is the zero potential contact with the hub. On the outside of the male fitting, a compressed spring forces the proximal catheter contact to mate with the male connection.

The electric leads to heat the catheter are connected through electric tab 57 and the hex nut 62 of threaded nipple 52. The leads could be attached by soldering, clipping or other convenient means.

As an example, heating of the electrodag coating is achieved by applying 24 volts at 4.5-6 amps to the leads for 7 seconds, and then applying 12 volts at 3 amps for the remainder of the heating cycle which is indicated by the ability to remove the catheter from the endoscope (approximately 13-15 seconds depending on the cryo-treatment exposure time). No negative effects will occur if the heater is applied longer than these time frames. The resistance of the electrodag generates heat as the current is passed through the length of the catheter. The initial 24 volts provide a quick initial thaw, while the remaining heating phase maintains and finishes the thaw cycle. All materials maintain all structural and functional properties through the entire heating cycle.

The herein disclosed invention has been described in terms of a catheter with an electrodag coating, however, other electrical means such as a conductive powder coating, a catheter made of conductive plastic or the like or metal would be operative.

In a broad aspect, the herein disclosed invention envisions a heated catheter in combination with an endoscope, with the heated catheter being fitted into a lumen of the endoscope.

Following a treatment cycle, the catheter may be warmed by depressing the right side of the foot pedal. A light on the solenoid will indicate that the catheter is experiencing a thaw cycle. The cycle can be interrupted at any time by releasing the foot pedal. Heat is generated by the resistance in the electrodag coating applied to the outer surface of the catheter. The hub has two contacts. The internal contact extends from the hub, through the catheter's internal surface to the distal fitting by means of the internal copper wire. This lead maintains a zero potential at all times. The second contact is located on the top surface of the hub. This contact extends from the hub, through the copper foil along the exterior of the catheter and into the proximal electrodag coating. Advantages of the Heated Catheter

The heated catheter provides a number of advantages over a traditional catheter:

Polyimide or PTFE, the Cryo-catheter material base, acts as a strong insulator and transports the liquid nitrogen with minimal thermal temperature loss resulting in a shorter time to achieve the clinically required cryoburn.

The heating mechanism allows the catheter to be removed from the endoscope lumen immediately following the cryo-therapy. More specifically, using a traditional catheter, the catheter is frozen into the endoscope lumen for 1-5 minutes following the therapy. This freezing to the endoscope lumen may result in damage to the endoscope.

Insulated Fittings

The new fittings on the device will be vacuum insulated. This will keep the fittings from frosting or feeling super cool to the human touch.

In addition, the hub or connective fittings which couple the catheter to the cryosystem have been redesigned and improved to accommodate electrical contacts required for the heating system.

### Alternative Embodiments of the Heated Catheter

An alternative embodiment is a heating coil on the heated catheter being energized in “series” or heated with a continuous length of wire energized from two ends. Also contemplated is a catheter with the heating element in parallel. This will result in heating short segments (5-10 segments per catheter) quickly and with more energy. The wrappings of the heating coil may be adjusted so that the loops touch one another. A parallel electrical transfer may be necessary. It may be feasible to employ flat wire (square wire) as opposed to round wire. Whether to use series or parallel spacing will be determined based on individual use. Coating the gap between the wires with a heat sink which may act to absorb radiated heat from the heating coil to dispense the heat to the outside of the catheter. A spray coat or liquid paint of a nichrome conductor may also be used. In this embodiment the entire catheter could be energized quite quickly. Alternate means may also be used for diverting freezing temperatures from non-target areas. Examples of such diverting means is a polystyrene tape to function as an insulator. Alternatively, the catheter may be made of polystyrene or some other insulating material. During the cryoburn the heat of the catheter remains active. This prevents the accidental injury to non-target tissue.

A further alternative embodiment of a heated catheter is a composite constructed of three different materials; in three different layers. The catheter itself (as the first layer) is made of extruded polyimide. Surrounding the first layer (the catheter) is a layer of magnetic wire wrapped around the outer diameter of the polyimide catheter. As a top or final layer, there is supplied a thin polyester heat shrink.

More specifically, the heated catheter (cryocatheter) can be defined as an extruded polyimide tube (O.D. 0.092″). Over the catheter is wrapped a layer of magnetic copper wire (0.007″ diameter). A number of different diameter wires are available. Use has been made of prototypes with 0.003″ diameter wire, 0.002″ diameter wire, 0.005″ diameter wire, etc. A 0.007″ diameter wire was the best for the desired voltage, but the invention does not exclude the use of wires of other diameters.

The wrappings of wire that functioned the best were 8 wraps per inch (a single strand was run the length of the catheter, and the wrapping was applied back over this single strand to complete the electrical loop. Double strand wrapping with the wrap spacing (up to 25 wraps per inch) would be operative.

A selected preferred voltage for application is 12 volts and 1 amp. Voltages of 5, 12, 17 and 24 volts have been tested. The important thing to keep in mind is that different diameter wires work well if wrapped to the correct density and heated with the appropriate amount of voltages.

The final layer employed is a thin (0.00025″) polyester heat shrink. This heat shrink serves to hold the wire in place and to seal the wire from patient contact.

The following examples are presented to further demonstrate application and use of the herein described methods and apparatus.

### EXAMPLE 1

### Pulmonary Cryospray Ablation for Treatment of Benign Airway Disease

The cryospray was used in the present studies to demonstrate the utility of the present invention for use in the treatment of lesions of the tracheobronchial system. In particular, the present example demonstrates the utility of the present invention for treatment of benign airway disease (BAD) of the lung using liquid nitrogen sprayed through a catheter via a flexible fiber optic bronchoscopy (FFB).

The present study will identify an effectiveness endpoint as an improvement in luminal patency following cryospray treatment along with visual confirmation of an absence of scarring and stricturing of the airway. If after the initial repeat bronchoscopy, it is determined that there is no immediate need for further intervention, then any future bronchoscopy will be performed upon the patient presenting with symptoms.

The cryospray ablation system will be used in the present studies. This system is a cryosurgical device that utilizes a low-pressure liquid nitrogen spray tip CSATM catheter medical grade liquid nitrogen will be used as the cryogen in the device. The device is used to destroy unwanted tissue by the application of extreme cold with the focused application to select tissue. The cryogen is stored in a liquid nitrogen holding tank integrated into the system.

The present study will consist of up to 10 subjects with benign airway disease. Treatment dosimetry will be up to 4, 5-second spray cycles. Subjects will have initial cryospray treatment at Day 0. Subjects will undergo repeat bronchoscopy in the first three to seven days after the initial treatment, to check for mucosal sloughing and to reassess luminal patency of the airway. Subjects may undergo up to one bronchoscopy per week with CSA therapy for a total of four (4) treatments in the first month. If they present with symptoms thereafter, then a repeat bronchoscopy will be performed; if luminal obstruction is noted, then the subject will begin the treatment protocol again. If disease exists bilaterally, only one side will be sprayed initially.

These studies on living swine, which are a valid model of the human tracheopulmonary system, establish that cryotherapy spray of liquid nitrogen via upper endoscopy is a useful ad effective technique capable of inducing controlled superficial mucosal damage with complete healing in the trachea and/or bronchial tissue.

The low-pressure device (FIGS. 1-26) described by Johnston (Gastrointest. Endoscop. 1999) and colleagues, uses liquid nitrogen in a specially designed system that operates at a maximum of 30 psi. The catheter, 10F, is multilayered. Its outer sheath is coated with a special polymer that can be warmed during the cryo application, thus maintaining catheter pliability and the unique ability to operate at a very low pressure. This device also uses a foot pedal for control of gas release and a temperature probe for monitoring mucosal temperature during the cryo application. With this delivery system, the depth of injury is controlled by manipulating 3 parameters: the duration of cryo application, extent of cryoburn viewed endoscopically, and the temperature of mucosa at the time of application. These parameters are monitored via a special software program and device that is part of the cryogenic system.

Low Pressure Cryo-therapy Device. Phases of animal research will be conducted with the low-pressure device. In the first phase, twenty swine will undergo cryoablation of the distal 2-3 cm of their tracheobronchial pathway with liquid nitrogen in either a hemi-circumferential or circumferential pattern and were followed for one month post-cryotherapy (Johnston, Gastrointest. Endosc. 1999). In the second phase, swine will be treated hemi-circumferentially to the distal 3 cm of the tracheobronchial pathway and followed for 90 days. In the third phase of studies, swine will undergo endoscopic ultrasound (EUS) of their tracheobronchial pathway, pre-cryo, immediately post cryo and then at 48 hours, 7 days and 14 days to assess the effects on the tracheopulmonary tissue wall. In the final phase, swine may be treated in different pulmonary locations with Argon plasma coagulation (APC), Multi-polar electrocoagulation (MPEC) and cryotherapy. The lesions may then be compared endoscopically and microscopically.

Primary outcome measures the effectiveness endpoint used will be observations of an improvement in luminal patency following cryospray treatment along with confirmation of an absence of scarring and structuring of the airway. The primary safety endpoint will include the reporting of all adverse events.

The proposed study is a single center pilot study consisting of up to 10 subjects with benign airway disease. Treatment dosimetry will be up to 4, 5-second spray cycles. Subjects will have initial cryospray treatment at Day 0. Subjects will undergo repeat bronchoscopy in the first three to seven days after the initial treatment, to check for mucosal sloughing and to reassess luminal patency of the airway. Subjects may undergo up to one bronchoscopy per week with CSA therapy for a total of four (4) treatments in the first month. If they present with symptoms thereafter, then a repeat bronchoscopy will be performed; if luminal obstruction is noted, then the subject will begin the treatment protocol again. If disease exists bilaterally, only one side will be sprayed initially.

Eligibility: Ages Eligible for Study: 18 Years and older Genders Eligible for Study: Both.

Criteria: Inclusion Criteria: 18 years of age, deemed a candidate for cryotherapy based on physician physical or medical history review, deemed operable based on institutional criteria.

Exclusion Criteria: Pregnant or nursing; planning to sire a child while enrolled in the study; known history of unresolved drug or alcohol dependency that would limit ability to comprehend or follow instructions related to informed consent, post-treatment instructions, or follow-up guidelines; refusal or inability to give consent; concurrent chemotherapy; prior radiation therapy which involved the any area between the vocal chords and the diaphragm; medical contraindication or potential problem that would preclude study participation.

Secondary Outcome Measures: A measure of treatment durability. If after the initial repeat bronchoscopy, it is determined that there is no immediate need for further intervention, then any future bronchoscopy will be performed upon the patient presenting with symptoms.

### EXAMPLE 2

### Interventional Cryotherapy in the Pleural Space

The present example demonstrates the utility of cryospray ablation to treat neoplastic lesions on the parietal pleural surface. Condition: Primary malignancy with metastasis to the parietal pleura pleural surface neoplasms; Intervention: Device: cryospray ablation system.

Primary Outcome Measures: To reduce tumor burden in the pleural space, as determined by visual inspection and biopsy of the treatment sites 2-5 days post treatment. Safety endpoint clinical and radiographic status at 30 days post cryospray treatment and adverse events. Secondary Outcome Measures: To determine if cryospray causes a pleurodesis effect. To determine if cryospray affects production of malignant effusion within the treated pleural cavity. To determine if pleural cavity treatment with cryospray is dosimetry dependent. Intervention Details: Device: Cryospray Ablation System.

Subjects will be treated with CryoSpray therapy at Day 0 using up to 3 cycles of 1040 second sprays as the studied dosimetry and will cover the affected area, including the tumor and the parietal pleural surface. If disease exists bilaterally, only one side will be sprayed. Subjects will be assessed 2-5 days from the initial treatment (Day 0) to check for mucosal sloughing, to reassess tumor burden and for additional CSA therapy as needed.

The proposed study is a pilot study consisting of an estimated 10 subjects with biopsy-proven metastatic cancer in the parietal pleural surface treated with CryoSpray at dye marked metastatic foci.

Initial treatment dosimetry will be up to 3 cycles of 10-40 second sprays. If necessary, dosimetry changes may occur after 3 subjects are treated and observed. It is unknown if dosimetry will need to be increased to enhance effectiveness. The PI may increase dosimetry by 1 cycle after each subject is treated and observed as a conservative approach to efficacy determination. If disease exists bilaterally, only one side will be sprayed.

Eligibility: Ages Eligible for Study: 18 Years and older Genders Eligible for Study: Both.

Criteria: Inclusion Criteria: 18 years of age; deemed a candidate for cryotherapy based on physician physical or medical history review; deemed operable based on institutional criteria; able to sign informed consent; documented lung or other visceral cancer with pleural involvement; WBC>4,000/mm3, platelets>100,000 mm3; physically well enough to undergo moderate sedation and pleuroscopy; female patients must be HCG negative; there should be direct evidence of disease progression despite treatment in previously treated patients.

Exclusion Criteria: Pregnant or nursing; planning to sire a child while enrolled in the study; known history of unresolved drug or alcohol dependency that would limit ability to comprehend or follow instructions related to informed consent, post-treatment instructions, or follow-up guidelines; refusal or inability to give consent; concurrent chemotherapy; medical contraindication or potential problem that would preclude study participation; concurrent participation in other experimental studies; uncontrolled coagulopathy or bleeding diathesis.

### EXAMPLE 3

### CryoSpray Abalation in Malignant Airway Disease

The present example demonstrates the utility of CryoSpray Ablation “CSA” or “cryospray therapy” to treat malignant airway disease in lung using liquid nitrogen sprayed through a catheter via flexible fiber optic bronchoscopy (FFB). The conditions that may be treated according to the present methods include lung cancer, mesothelioma, and malignant airway obstruction, among others.

Measures: Efficacy of the cryogen on a tumor evaluated by histopathological data and visual inspection along with visual confirmation of absence of scarring and stricturing of the airway. The primary safety endpoint is the reporting of all adverse events. Designated as safety issue: Yes Secondary Outcome Measures: Consists of a measure of treatment efficacy and improvement in luminal patency assessed by visual inspection. Treatment dosimetry will be up to 4, 5-second spray cycles. Subjects will have initial cryospray treatment at Day 0. Subjects will undergo repeat bronchoscopy in the first seven days after the initial treatment to check for mucosal sloughing and to reassess luminal patency of the airway. Subjects may undergo up to one bronchoscopy with CSA therapy every seven days for a total of four (4) treatments in the first month. If they present with symptoms thereafter, then a repeat bronchoscopy will be performed; if luminal obstruction is noted, then the subject will begin the treatment protocol again. Subjects may also have rigid/flexible bronchoscopy with laser or electrocautery snare for debulking of tumors. If disease exists bilaterally, only one side will be sprayed initially.

The primary endpoint is efficacy of the cryogen on the tumor evaluated by histopathological data and visual inspection along with visual confirmation of an absence of scarring and stricturing of the airway. The primary safety endpoint is the reporting of all adverse events. The primary symptom measures are the St. Georges Respiratory Questionnaire (SGRQ) and the Borg Dyspnea Index (BDI).

The secondary endpoint will consist of a measure of treatment efficacy and improvement in luminal patency assessed by visual inspection. If after the initial repeat bronchoscopy, the investigator determines that there is no immediate need for further intervention, then any future bronchoscopy will be performed upon the subject presenting with symptoms.

The proposed study will take place at up to three centers consisting of up to a total of 30 subjects with malignant airway obstruction. Treatment dosimetry will be up to 4, 5-second spray cycles Subjects will have initial cryospray treatment at Day 0. Subjects will undergo repeat bronchoscopy in the first seven days after the initial treatment to check for mucosal sloughing and to reassess luminal patency of the airway. Subjects may undergo up to one bronchoscopy with CSA therapy every seven days for a total of four (4) treatments in the first month. If they present with symptoms thereafter, then a repeat bronchoscopy will be performed; if luminal obstruction is noted, then the subject will begin the treatment protocol again. Subjects may also have rigid/flexible bronchoscopy with laser or electrocautery snare for debulking of tumors. If disease exists bilaterally, only one side will be sprayed initially.

The study population consists of up to 30 subjects with malignant airway obstruction as a consequence of an endoluminal tumor that obstructs a portion of the respiratory tree below the vocal chords. These subjects will have been apprised of Standard of Care options, and will have rejected those options or have been deemed ineligible for them. Subjects must have a signed consent form and satisfy all study inclusion and exclusion criteria.

It is estimated that enrollment will take approximately 6 months. Each subject will receive CryoSpray treatments over the course of 1 year.

Eligibility: Ages Eligible for Study: 18 Years and older Genders Eligible for Study: Both.

Inclusion Criteria: 18 years of age or greater; deemed a candidate for cryotherapy based on physician physical or medical history review; deemed inoperable based on institutional criteria.

Exclusion Criteria: 18 years of age or greater; Pregnant or nursing; planning to sire a child while enrolled in the study; known history of unresolved drug or alcohol dependency that would limit ability to comprehend or follow instructions related to informed consent, post-treatment instructions, or follow-up guidelines; refusal or inability to give consent; concurrent induction chemotherapy; radiation therapy within the last 30 days which involved the any area between the vocal chords and the diaphragm; medical contraindication or potential problem that would preclude study participation; concurrent participation in other experimental studies; uncontrolled coagulopathy or bleeding diathesis; serious medical illness, including; uncontrolled congestive heart failure; uncontrolled angina; myocardial infarction; cerebrovascular accident within 6 months prior to study entry.

### EXAMPLE 4

### CryoSpray Ablation in Creating a Thoracic Patient Registry

The present example provides a method by which a collection of patient material will be gathered and analyzed who have been treated with a cryogenic method according to the present invention.

The following pulmonary conditions will be identified in each subject admitted as part of the registry: Lung Cancer, Emphysema, Chronic Bronchitis COPD, Asthma, Sarcoidosis, Mesothelioma.

The primary objective for creating the registry is to enable analysis of patient outcomes 2 years following final treatment and to estimate the effectiveness of the device in eradicating, decreasing and downgrading of pulmonary diseases.

### EXAMPLE 5

### CryoSpray Ablation of Surgical Resection Specimens in Lung Cancer Patients

The purpose of the present example is to provide a framework within which to evaluate the feasibility and general safety of cryoablation as a treatment regimen in the human airway as well as assess the safety and depth and area of treatment using liquid nitrogen sprayed through a catheter via flexible fiber optic bronchoscopy (FFB) using surgical resection specimens from patients undergoing lobectomy.

Condition: Lung cancer.

Intervention: Device: cryospray ablation system.

The histological effects of cryospray therapy in lobectomy patients will be identified in the present studies.

Eligibility: Ages Eligible for Study: 18 Years and older Genders Eligible for Study: Both.

Criteria: Inclusion Criteria: Age 18 years of age; lobectomy planned based on clinical situation not related to this study; deemed operable based on institutional criteria.

Exclusion Criteria: (See Example 1).

### EXAMPLE 6

### Cryoablation Device

The cryosurgical apparatus of the present invention is as described in U.S. Pat. No. 6,027,499, U.S. Pat. No. 7,025,762, U.S. Pat. No. 7,255,693 and US Published Patent Application 2007/0276360. The contents of each of these patents is specifically incorporated herein by reference.

The cryosurgical apparatus may in some embodiments be modified so as to include a distal end dimensioned for insertion into a patients trachea. In this manner, the cryosurgical apparatus may be used in the cryoablation of tracheal tissue, lung tissue, or any desired portion or focused area in a patients tracheobronchial pulmonary system.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

### BIBLIOGRAPHY

All of the following documents are specifically incorporated herein by reference.
U.S. Pat. No. 6,027,499
U.S. Patent Application 2007/0276360A1
U.S. Pat. No. 7,025,762
U.S. Pat. No. 7,255,693
Johnston M H Z, Easton J A, Hardhat J D, Cart ledge J, Mathews J S, Foggy J R. Cryoablation of Barrett's esophagus: a pilot study. Gastrointest Endosc. December 2005;62(6):842-8.
Johnston C M, Schoenfeld L P, Mysore J V, Dubois A. Endoscopic spray cryotherapy: a new technique for mucosal ablation in the esophagus. Gastrointest Endosc. July 1999:50_{—}01:86-92.
Ell C May A, Gossner L, Pech O, Gunter E, Mayer G, Henrich R, Vieth M,_Muller H, Seitz G, Stolte M. Endoscopic mucosal resection of early cancer and high-grade dysplasia in Barrett's esophagus. Gastroenterology. April 2000;118(4):670-7.
Champion G, Richter J E, Vaezi M F, Singh S, Alexander R. Duodenogastroesophageal reflux: relationship to pH and importance in Barrett's esophagus. Gastroenterology. September 1994;107(3):747-54.
Eisen G M, Sandler R S, Murray S, Gottfried M. The relationship between gastroesophageal reflux disease and its complications with Barrett's esophagus. Am J Gastroenterol. January 1997;92(1):27-31.
Johnston M H. Cryotherapy and other newer techniques. Gastrointest Endosc Clin N Am. July 2003;13 (3):491-504. Review.
Cash B D, Johnston L R, Johnston M H. Cryospray ablation (CSA) in the palliative treatment of squamous cell carcinoma of the esophagus. World J SurLOncol. Mar. 16, 2007:5:34.
Field J K, Youngson J H. The Liverpool Lung Project: a molecular epidemiological study of early lung cancer detection. Eur Respir J. August 2002:20(2):464-79.
Pinsonneault C, Fortier J, Donati F. Tracheal resection and reconstruction. Can J Anaesth. May1999;46(5 Pt 1):439-55. Review.
Johnston M H, Horwhat J D, Haluska, Moses F M. Depth of injury following endoscopic spray cryotherapy: EUS assisted evaluation of mucosal ablation and subsequent healing in the swine model (Abstract). Gastrointestinal Endoscopy 51: AB98, 3462, 2000.
Eastone J A, Horwhat D, Haluska O, Mathews J, Johnston M. Cryoablation of swine esophageal mucosa: A direct comparison to argon plasma coagulation (APC) and multipolar electrocoagulation (MPEC) [Abstract] Gastrointestinal Endoscopy 53: A3448, 2000.
Johnston M H, Eastone J A, Horwhat J D. Reversal of Barrett's esophagus with cryotherapy [Abstract]. American Journal of Gastroenterology 98(9 Suppl): A30, S11, 2003.
Johnston M H, Cash B D, Horwhat J D, Johnston L R, Dykes C A, Mays H S. Cryoablation of Barrett's Esophagus (BE) [Abstract]. Gastroenterology 130 (4, Suppl.2): A640, 2006.
Johnston M H, Cash B D, Dykes C A, Mays H S, Johnston L R. Cryoablation of dysplasia in Barrett's Esophagus (BE) and early stage esophageal cancer [Abstract]. Gastrointestinal Endoscopy 63 (5): April, 2006.

The invention is claimed as follows:

1. A method for treating a tracheobronchial system comprising:
a.) providing a cryosurgical apparatus having a distal end dimensioned for insertion into a patients trachea, said cryosurgical apparatus comprising:
an elongated, flexible tubular body having a proximal end, a distal end and a tubular passageway extending there through for carrying a cryogen from the proximal end to the distal end, the proximal end being adapted to receive a cryogen, the distal end adapted to dispense cryogen to a desired area of the tracheobronchial system of a patient at a low temperature and low pressure, and
a cryogen source
b.) inserting the distal end of the apparatus into the trachea of the patient and positioning said distal end within an area of the tracheobronchial system of; and
c.) dispensing the cryogen to the area of interest in an amount and for a period of time sufficient to result in tissue ablation.

2. The method of claim 1 wherein the cryogen is provided as a spray to the area of interest in a substantially radial direction and substantially perpendicular to the axis of the catheter.

3. The method of claim 1 wherein the area of interest comprises an area having a tracheobroncheal lesion, a vascular lesion, a neoplastic degenerative lesion or an inflammatory lesion.

4. The method of claim 1 wherein the cryosurgical apparatus comprises a bronchoscope dimensioned for insertion into a patients trachea having a scope length of about 870 mm, a channel inner diameter of about 2.0 to about 3.0 mm, and a distal outer diameter of about 5.0 mm. to about 6.0 mm.

5. The method of claim 1 wherein the tracheobronchial system area of interest comprises a diseased area comprising benign airway disease, early lung cancer, sarcoidosis, Wegner's granulomatous, rhinoscleroma, recurrent respiratory papillomatosis (RRP), benign tracheal stenosis, pulmonary dysplasia, neoplasia, vascular lesions, inflammatory lesions, pulmonary degenerative lesions, lung cancer, emphysema, chronic bronchitis, COPD, asthma or mesothelioma.

6. A cryosurgical apparatus for cryogenic spray ablation, comprising:
a cryogen source;
an endoscope dimensioned to be inserted within a patient's trachea; and
a catheter carried by the endoscope and connected to said cryogen source by a conduit, said catheter having a distal end through which a cryogenic spray is applied to the patient's trachea,
wherein the apparatus is configured such that, in use, low temperature, low pressure substantially liquefied cryogen exits the catheter distal end, and wherein the distal end of the catheter is adapted to spray the cryogen in a radial direction substantially perpendicular to the axis of the catheter.

7. The cryosurgical apparatus of claim 6, comprising an electronically-controlled timer.

8. The cryosurgical apparatus of claim 7, wherein the electronically-controlled timer includes a print-out that is patient specific.

9. The cryosurgical apparatus of claim 6, wherein cryogen is delivered substantially instantaneously to the conduit at a pressure suitable for cryogenic spray ablation.

10. The cryosurgical apparatus of claim 6 further comprising a means for viewing the area to be ablated.

11. The cryosurgical apparatus of claim 6 wherein the viewing means is disposed together with an illuminating light source and imaging camera.

12. The cryosurgical apparatus of claim 6 further comprising an inflating means.

13. The cryosurgical apparatus of claim 6, wherein the distal end of the catheter is adapted to spray the cryogen in a radial direction substantially perpendicular to the axis of the catheter by bending.

14. The cryosurgical apparatus of claim 6, wherein a heating device is attached to the cryogen source to aid in the efficient delivery of said cryogen for ablation.

15. The cryosurgical apparatus of claim 6, further comprising a foot-pedal controlled solenoid-actuated valve between the catheter and the cryogen source.

16. A method for treating a tracheobronchial internal lesion comprising:
providing the cryosurgical apparatus of claim 6; and
applying a cryogen to said lesion, thereby ablating said lesion.

17. The cryosurgical apparatus of claim 6, further comprising an electronically-controlled timer provided with a display indicating the time interval that the cryogenic spray has been discharged from the distal end of the catheter,
